(19) United States
(12) Patent Application Publication
(10) Pub. No.: US 2015/0158750 A1
(43) Pub. Date: Jun. 11, 2015

(54) Water faucet with integrated contact and contamination protector and photocatalytic disinfection

(71) Applicant: Simon Schiffmann, Hamburg (DE)

(72) Inventors: Simon Schiffmann, Hamburg (DE); Wolfgang Schiffmann, Kaltenkirchen (DE)

(21) Appl. No.: 14/556,175

(22) Filed: Nov. 30, 2014

(30) Foreign Application Priority Data
Dec. 5, 2013 (DE) 202013010964.5

(51) Int. Cl. C02F 1/72; A61L 2/10; E03C 1/10; C02F 1/32

(52) U.S. Cl. CPC C02F 1/725; C02F 1/32; A61L 2/10; E03C 1/10; C02F 2307/06; C02F 2305/02; C02F 2305/10; C02F 2303/04; C02F 2201/326; E03C 2201/40

(57) ABSTRACT

A water faucet for the supply of germ-free water for human consumption comprises a faucet housing which is spatially separated from a water pipe that is arranged within the faucet housing. A contact protector with overflow openings is provided on a water outlet end of the faucet housing. The overflow openings prevent contaminated water from entering into the faucet, even if the water outlet of the faucet were to be blocked. An aerator is fastened on a discharge end of the water pipe inside the faucet housing. An air-gap is provided between the aerator and the water outlet opening of the contact protector. Internal surfaces of the faucet housing and of the contact protector are coated with a photocatalytic material such as titanium dioxide and are exposed to UV-A light from an ultraviolet light source while and after water is flowing through the water pipe.

### TECHNICAL FIELD

The present disclosure generally relates to water faucets, and particularly to a water faucet with an integrated contact and contamination protector and photocatalytic disinfection of the internal housing thereof.

### BACKGROUND

The use of water faucets of any type does not preclude a human contact. Even in a contactless water flow arrangement, there is a possibility of contacting the faucet or water-pipe by contaminated objects, e.g. the hands of a human user. Due to such possible contact, germs present on hands can easily be transmitted to water faucet interior surfaces and thus reach the biofilm of a water systems through the water faucet discharge or outlet. In addition, contamination of the water faucet surfaces and thereby of the biofilm happens, as indicated above, when these come in contact with the body secretions caused by coughing, sneezing etc.

Previous approaches to provide largely germ-free water for human consumption included various technologies (e.g. UV-C disinfection, carbon filter, etc.) for treating water before being discharged from the faucet for human consumption. However, even after using these technologies, contamination of the biofilm could still be possible due to contamination of water in the areas beyond such previously available systems, and the delivery of completely germ-free water from the water faucet cannot be guaranteed.

Various studies have been carried out to address this existing problem, prevailing until now, of the contamination of water supply used for human consumption. It was noted that the biofilm present in water system represents the boundary layer between the water and the water supply (water-pipe), in which nutrients and other substances are present in a dissolved form. The presence of germs in the biofilm may pose serious health hazards to humans. So, it necessitated a need to look for new systems and techniques, which can either stop the contamination of the biofilm in the water delivery system or which can decompose the harmful germs, organic compounds or organisms present in the biofilm.

In recent times, the photocatalytic phenomenon has been studied intensively now for many years by may groups, and it is fairly well understood. Because TiO₂ (Titanium dioxide) is a semiconductor with a band gap of about 3.0 eV, UV light (with wavelengths shorter than 400 nm) can excite pairs of electrons and holes, as already discussed. The photogenerated electrons then react with molecular oxygen (O2) to produce superoxide radical anions (_O2—), and the photogenerated holes react with water to produce hydroxyl (—OH) radicals. These two types of rather reactive radicals then work together to decompose the organic compounds. The longer the film is illuminated with UV light, the more organic material can be decomposed, so that, for example, an oily stain on the surface would gradually disappear.

[Source: “Titanium dioxide photocatalysts” by Akira Fujishima, Tata N. Rao, Donald A. Tryk published in the Journal of Photochemistry and Photobiology C: Photochemistry Reviews 1 (2000) 1-21].

Further, in the abstract of the article “Bacterial Effects of Cold Sprayed Titanium Dioxide Coatings” by J.-O. Kliemann, H. Gabriel, H. Gutzmann, F. Gartner, T. Klassen published in Keramische Zeitschrift January 2011, it was cited that—Titanium dioxide in anatase phase structure has high antibacterial activity due to its photocatalytic activity. For this study, ceramic TiO₂ coatings on stainless steel were produced by cold spraying. The bacterial effect of the coatings was tested with Pseudomonas aeruginosa bacteria. A kill rate of 99.9% on UV-A light was already achieved after 5 minutes. While the raw stainless steel reference did not show any significant reduction even after 60 minutes. The results reveal that cold sprayed Titanium dioxide coatings can serve as self-disinfecting surfaces.

It was further established by the Fraunhofer-Allianz for Photocatalysis of Braunschweig, Germany, that photocatalytic and photohydrophilic TiO₂ coatings can be employed on surfaces for:
Reducing the efforts for cleaning;
Self-cleaning;
Self-disinfection;
Anti-fog/mist effect;
Hydrophilic treatment; and
Gas and liquid purification.

In particular, the crystalline TiO₂ is a material having an excellent potential for innovative products and processes, e.g. for photocatalysts reactions, in which, the impingement of the coating by means of UV-A radiation or sunlight after activation of TiO₂, oxidation and reduction processes are stimulated. This also leads to decomposition of organic and inorganic substances on the target surfaces.

Accordingly, in the present field of application, an antimicrobial effect can be used effectively for destructing or decomposing the microorganisms overlaying the target surface thanks to their decomposition reactions (without using any chemical substances, e.g. antibiotics). This particular characteristic of TiO₂ is used in accordance with the present invention, as outlined in the description below.

### SUMMARY

The present invention eliminates the existing problem, in which the water supply delivered from faucets and used for human consumption can become contaminated. With prior art faucets such contamination occurs when the faucet or water pipe comes in contact with contaminated objects, e.g. hands, which carry germs or other harmful organisms. These germs or organisms can be easily transmitted to internal surfaces of a known faucet and/or water pipe, and can reach the biofilm of a water supply systems after entering through a water outlet openings of a traditional faucet.

The present disclosure provides a water faucet with integrated contact and contamination protector, which is configured to prevent transmission of germs/biological organisms into water systems which supply water for human consumption. This is achieved by spatially separating an internal water pipe in the faucet from an externally accessible faucet housing and faucet water discharge or outlet opening. A constructional arrangement is provided to protect the internal faucet components from direct human contact so as to avoid contamination of the biofilm of the water system.

To further prevent possible contamination, any faucet surfaces that might become contaminated are not only physically separated from the water pipe which is connected to the water supply, but also photocatalytically disinfected. Photocatalytic disinfection is achieved by coating internal surfaces of the faucet with titanium dioxide (TiO₂) and exposing the so coated surfaces to UV-A light from an ultraviolet light source. This ensures that the transmission of germs/biological organisms into the water supply and thereby into the biofilm thereof is rendered impossible.

A water faucet for the supply of germ-free water for human consumption thus comprises a faucet housing which is spatially separated from a water pipe that is arranged within the faucet housing. A contact protector is provided on a water outlet end of the faucet housing. The contact protector has a water outlet opening and a plurality of overflow openings. The overflow openings prevent contaminated water from entering into the faucet, even if the water outlet of the faucet were to be blocked. An aerator is fastened on a discharge end of the water pipe inside the faucet housing. An air-gap is provided between the aerator and the water outlet opening of the contact protector.

The contact protector may comprise a substantially cylindrical outer wall and a funnel-shaped inner wall section extending downwardly to a water outlet opening. The water outlet opening is arranged inside and above a lower end of the substantially cylindrical outer wall or an annular extension thereof. The contact protector may be configured with at least one row of circumferentially spaced overflow openings.

The faucet is preferably touchless, using an electrically controlled valve to control the flow of water from a water supply to the water pipe. To destroy biological contaminants that may have entered the faucet, internal surfaces of the faucet housing and internal surfaces of the contact protector are coated with a photocatalytic material and are exposed to UV-A light from an ultraviolet light source while and after water is flowing through the water pipe. The ultraviolet light source comprises a plurality of light emitting diodes with a wavelength between 315 and 400 nm that are mounted circumferentially spaced on an LED carrier fixed between the aerator and the discharge end of the water pipe.

When in use, the faucet detects the presence of an object, such as hands, under the water outlet end of the housing by a proximity sensor. It then activates the ultraviolet light source and monitors the operational status of the ultraviolet light source. The faucet activates the flow of water through the electrically activated valve only if the ultraviolet light source is operational. This provides a safeguard which disables the faucet in case the ultraviolet light source is defective.

### BRIEF DESCRIPTION OF THE DRAWINGS

A water faucet with integrated contact and contamination protector and with photocatalytic disinfection of the faucet internal housing is described in terms of the exemplary embodiments as set forth in the drawings, in which:

FIG. 1 illustrates a sectional view of an exemplary water faucet which is fitted over a sink or wash basin mounted on a fixed platform.

FIG. 2 illustrates a detailed view of a preferred embodiment of a contact protector, which is screwed onto the faucet housing.

FIG. 3 illustrates a detailed view of an alternative embodiment of the contact protector.

FIG. 4 is an enlarged detailed view of an outlet section of a faucet as in FIG. 1.

FIG. 5 illustrates schematically surfaces of the faucet which are coated with TiO₂.

### DETAILED DESCRIPTION

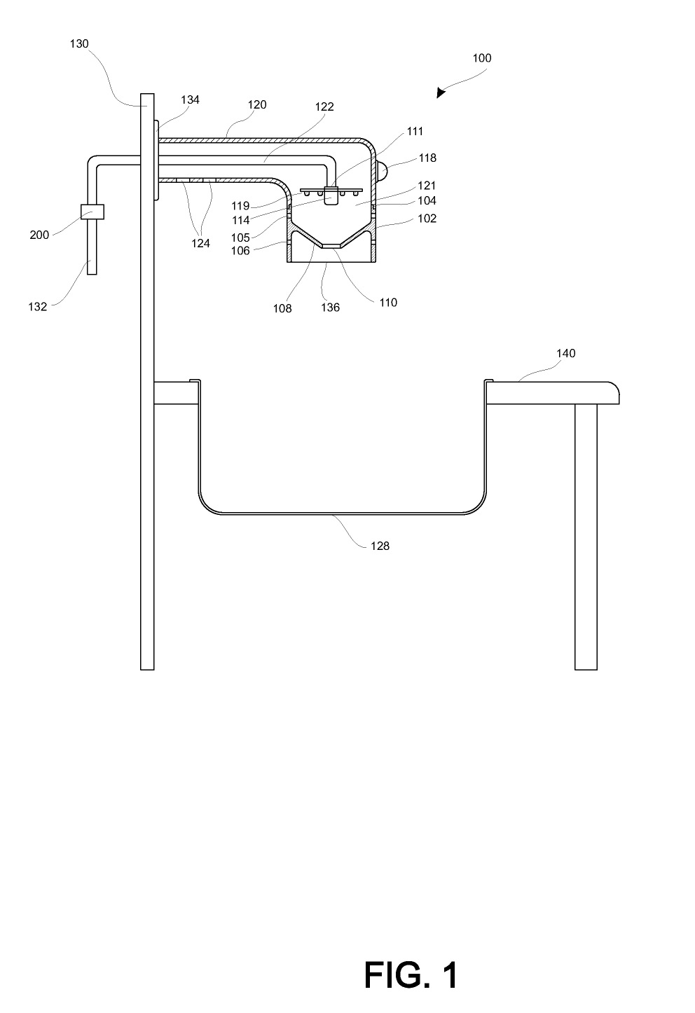

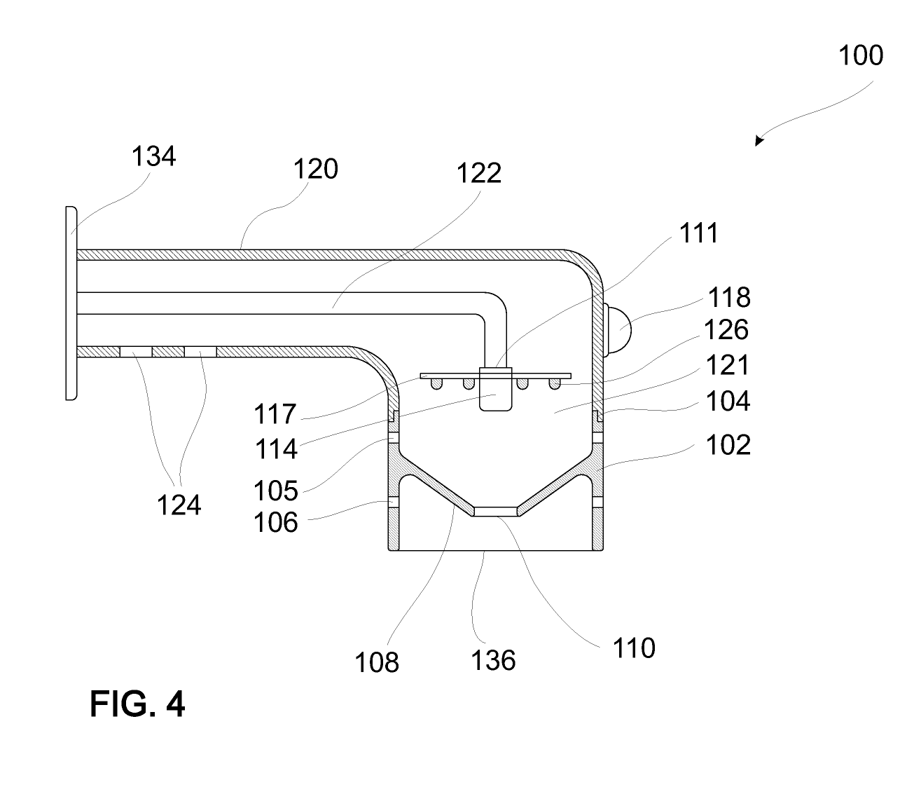

Referring to FIG. 1 and FIG. 4, a sectional view of a water faucet 100 is fitted over a sink 128 mounted on a platform 140. The faucet 100 comprises a faucet housing 120 which is fixed at a proximal end on a wall 130. A water pipe 122 is connected to a water supply 132 through an electrically activated valve 200. The water pipe 122 passes through the wall 130 at a base 134. The base 134 is firmly fixed to the wall 130. The water pipe 122 draws water from the water supply 132 when the electrically activated valve 200 is open. Water then flows through the water pipe 122 from an inlet end at the base 134 towards and through a water pipe discharge end 111 and a faucet outlet 136. Two vent holes 124 are provided on the bottom side of the faucet housing 120 between the base 134 and the faucet outlet 136, proximal to the base 134.

The faucet 100 is configured with a contact protector 102 which is screwed onto the distal end 121 of the faucet housing 120 by means of a screwed joint 104. The contact protector 102 includes a plurality of overflow openings 105, 106 that are arranged on its circumference in two rows disposed at a suitable distance from each other. The contact protector 102 is configured also with a conical outlet funnel 108 that is slanting down towards a discharge opening 110. The internal surfaces of the conical outlet funnels 108 are coated with a photocatalytic coating 112, which is preferably titanium dioxide (TiO₂). The overflow openings 105, 106 may be arranged either only on one side of the conical outlet funnel 108 or on both sides thereof. As shown in FIG. 1, a plurality of upper overflow openings 105 is disposed above the conical outlet funnel 108, and a plurality of lower overflow openings 106 is disposed below the conical outlet funnel 108. Preferably, an aerator 114 is mounted onto the discharge end 111 of the water pipe 122 and centrally aligned with the discharge opening 110.

An LED carrier 117 which forms the base for an ultraviolet light source 119 is placed at the discharge end 111 of the water pipe, and may be held by the aerator 114 when the aerator 114 is screwed onto the discharge end 111 of the water pipe 122. The ultraviolet light source 119 may comprise one or more ultraviolet LEDs 126 which emit light in the UV-A spectrum of 400-315 nm wavelength. The ultraviolet LEDs 126 may be disposed at the lower side of the LED carrier 117. The LED carrier 117 is preferably centrally aligned above the conical outlet funnel 108 of the contact protector 102. The ultraviolet LEDs 126 are thus in direct view of the inner surface of the conical outlet funnel 108.

At least one infrared proximity sensor 118 is fitted just above the joint 104 on the outside of the faucet housing 120 facing away from the base 134 and towards a user of the faucet. The aerator 114 and the LED carrier 117 are preferably externally coated with the photocatalytic material TiO₂. This way, only contamination/germ-free water which is fit for human consumption gets delivered through the discharge opening 110 that is disposed at the lower center of the conical outlet funnel 108. This germ-free water subsequently passed through the faucet outlet 136. The waste water flows down in the sink 128 and passes through an outlet (not shown) in the bottom of the sink 128.

The contact protector 102 of the faucet 100 has no direct mechanical connection with the internal water pipe 122 and with water supply 132 at any point downstream of the base 134. When in use, water flows out of the discharge opening 110 only after passing an air gap provided between the lower end of the aerator 114 and the discharge opening 110.

The contact protector 102 comprises a plurality of overflow openings 105, 106 and internal photocatalytically coated surfaces 112. The photocatalytic coating 112 in conjunction with the ultraviolet light source 119 ensures disinfection of faucet's inner surfaces, whereby contamination is prevented. The ultraviolet light source 119 is located above the aerator 114. Because of water-jet impingement in the direction of the conical outlet funnel 108, no water spray possibly splashed from the aerator 114 can reach the ultraviolet light source 119 so that calcification or damage of the ultraviolet light source 119 is prevented.

One or more infrared proximity sensors 118 are used for touchless operation of the faucet 100. When an object is sensed by the infrared proximity sensor 118, an electronic control circuit activates the ultraviolet light source 119 and the flow of water through the electrically activated valve 200 in a contactless manner. The operating time of the ultraviolet light source 119 is controlled by the control circuit (not shown), which ensures a beam incidence time longer than the time of water discharge from the discharge opening 110 and faucet outlet 136. The operational status of the ultraviolet light source 119 can be monitored by the control circuit, for example by measuring the electric current through the ultraviolet light source 119. The flow of water can be interrupted, if a failure of the ultraviolet light source 119 is detected, for example if the electric current through the ultraviolet light source 119 is above or below a predetermined acceptable range. The predetermined acceptable range may be selected such, that a failure of 50% or more of the ultraviolet light source 119 stops the flow of water through the electrically activated valve 200 by a safety circuit inside the control circuit.

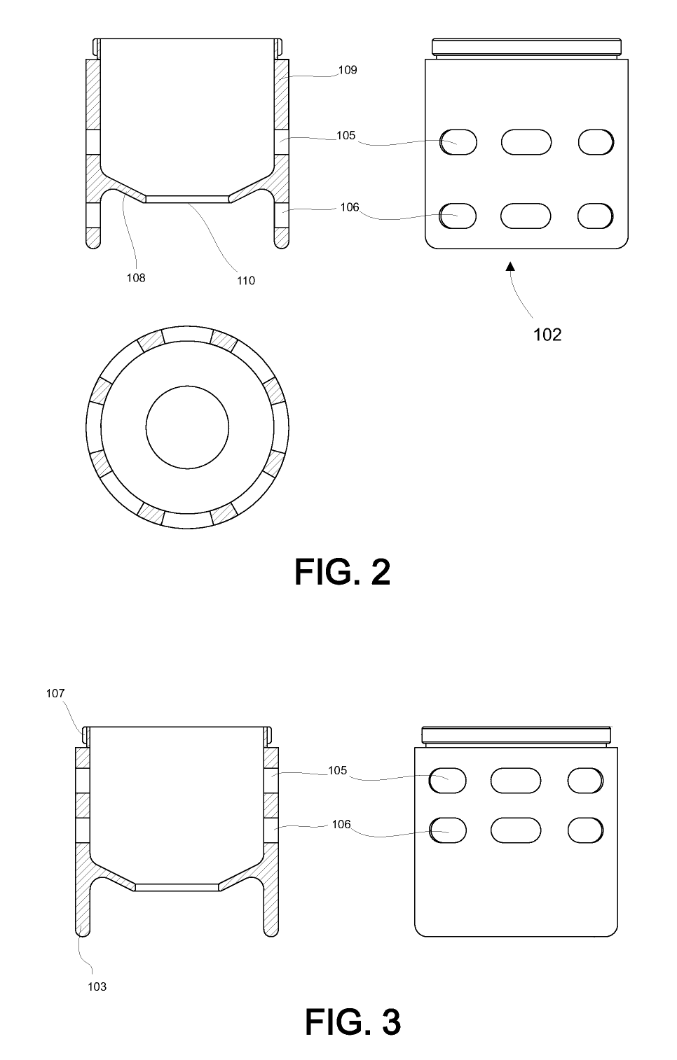

FIG. 2 illustrates a preferred embodiment of the contact protector 102 which may be attached to the faucet housing 120 by a screwed joint 104 configured at the upper end of the wall 109. The conical outlet funnel 108 with the discharge opening 110 at its lower end ensures contactless and thus, germ-free water discharge from water faucet 100. The wall 109 of the contact protector may extend downwardly into an annular extension 103 which is configured under the conical outlet funnel 108 and prevents a direct contact of objects, e.g. hands, with the discharge opening 110. Therefore, transmission of germs to a biofilm of the water supply 132 is prevented. In a first embodiment, the contact protector 102 includes screw threads 107 having a base and peaks to facilitate the screwed joint 104 with the water pipe 122. However, any other fastening arrangement may also be used by replacing the screwed joint 104 shown here.

The contact protector 102 also includes a plurality of overflow openings 105, 106, which are arranged in two rows disposed at a suitable distance from each other in the wall 109 of the contact protector 102. As shown in FIG. 2, the overflow openings 105, 106 may comprise a first plurality of upper overflow openings 105 disposed above the conical outlet funnel 108 and a second plurality of lower overflow openings 106 disposed below the conical outlet funnel 108. The cross-section of all overflow openings 105, 106 should be larger than the cross-section of the discharge end 111 of the water pipe 122, to ensure water discharge in the event of a blocked faucet outlet 136 and resulting back-pressure. Preferably, the cross-section of all overflow openings 105, 106 should be at least twice the cross-section of the discharge end 111 of the water pipe 122.

FIG. 3 illustrates a similar view of another preferred embodiment of the contact protector 102 of FIG. 2. Here, two rows of overflow openings 105, 106 are disposed above the conical outlet funnel 108.

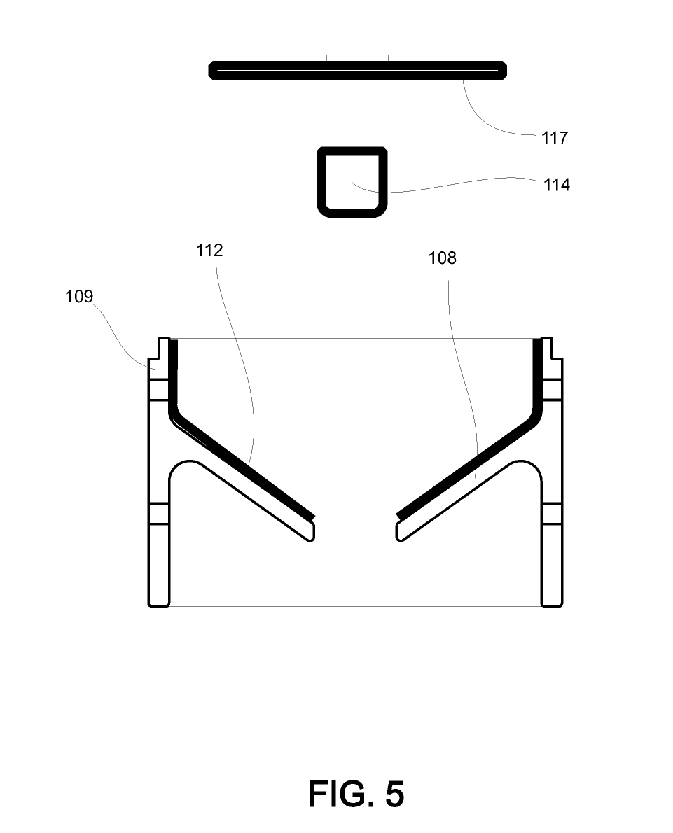

FIG. 5 illustrates schematically which surfaces of the faucet 100 are functionally disinfectant due to their photocatalytic coating 112. Those surfaces are shown in bold lines. In particular, the LED carrier 117, the aerator 114 and the inwardly facing surfaces of the contact protector 102, i.e. the conical outlet funnel 108 and the wall 109, are coated with titatinum dioxide (TiO₂).

### Operation of the Water Faucet

When in use, the infrared proximity sensor 118 detects the presence of an object, e.g. the hands of a human, under the faucet outlet 136. An electronic control circuit activates the ultraviolet light source 119 and monitors its operation. Only after the ultraviolet light source 119 is found to be operational, i.e. when the faucet is being disinfected and thus safe to discharge water for human consumption, the electrically controlled valve 200 is activated to start water flow through water pipe 122. Once the object moves away from under the faucet outlet 136, the valve 200 is closed, and water flow stops. In this case, the ultraviolet light source 119 is kept on for a predetermined time period until after the valve 200 is turned-off. The water jet issued from under the aerator 114 ensures that the water discharge from the faucet outlet 136 occurs only after passing the air gap. The contact protector's interior surfaces which are coated with a photocatalytic coating 112 and the and faucet housing interior are protected from any bacterial attack, as those are destroyed by the UV-A light from ultraviolet light source 119 in interaction with the photocatalytic coating 112. If the discharge opening 110 or faucet outlet 136 were to be blocked, water will flow through the overflow openings 105, 106 and ensure that even with a blocked faucet 100 contaminants cannot reach the upstream water supply.

While the present invention has been described with reference to exemplary embodiments, it will be readily apparent to those skilled in the art that the invention is not limited to the disclosed or illustrated embodiments but, on the contrary, is intended to cover numerous other modifications, substitutions, variations and broad equivalent arrangements that are included within the spirit and scope of the following claims.

What is claimed is:

1. A water faucet, comprising:
a base;
a water pipe extending from an inlet end at the base towards a discharge end;
an ultraviolet light source disposed at the discharge end of the water pipe; and
a housing surrounding the water pipe and the ultraviolet light source, the housing extending from the base to a water outlet end, the water outlet end being arranged below the discharge end of the water pipe,
wherein the housing is separated from the water pipe by an air gap, and
wherein an inner surface of the housing is coated with a photocatalytic coating.

2. The water faucet as in claim 1 wherein the housing does not contact the water pipe outwardly of the base.

3. The water faucet as in claim 1, wherein the photocatalytic coating is titanium dioxide.

4. The water faucet as in claim 1, further comprising a contact protector disposed at the water outlet end of the housing, wherein the contact protector comprises a conical outlet funnel having an inner surface that is coated with titanium dioxide, the conical outlet funnel being arranged concentrically below the discharge end of the water pipe in direct view of the ultraviolet light source.

5. The water faucet as in claim 4, wherein the contact protector comprises at least one overflow opening arranged above the conical outlet funnel.

6. The water faucet as in claim 4, wherein the contact protector comprises two or more circumferentially spaced overflow openings arranged above the conical outlet funnel, the overflow openings having a combined cross sectional area that is larger than a cross sectional area at the discharge end of the water pipe.

7. The water faucet as in claim 4, wherein the contact protector comprises an annular extension around and downwardly below the conical outlet funnel to prevent direct contact therewith.

8. The water faucet as in claim 7, wherein the annular extension comprises at least one overflow opening.

9. The water faucet as in claim 7, wherein the annular extension comprises two or more circumferentially spaced overflow openings having a combined cross sectional area that is larger than a cross sectional area at the discharge end of the water pipe.

10. The water faucet as in claim 1, further comprising:
a control circuit operatively connected to the ultraviolet light source;
a proximity sensor operatively connected to the control circuit; and
an electrically activated valve for controlling a flow of water through the water pipe, the electrically activated valve being operatively connected to the control circuit,
wherein the control circuit activates the ultraviolet light source and the flow of water through the electrically activated valve in response to a signal from the proximity sensor.

11. The water faucet as in claim 10, wherein the control circuit monitors operation of the ultraviolet light source and disables the flow of water through the electrically activated valve if a failure of the ultraviolet light source is detected.

12. The water faucet as in claim 10, wherein the control circuit keeps the ultraviolet light source activated for a predetermined time after disabling the flow of water through the electrically activated valve.

13. A water faucet for a supply of germ-free water for human consumption, comprising:
a faucet housing;
a water pipe arranged within the faucet housing;
an electrically controlled valve configured to control a flow of water from a water supply to the water pipe;
a contact protector fastened on a water outlet end of the faucet housing, the contact protector having a water outlet opening and a plurality of overflow openings;
an aerator fastened on a discharge end of the water pipe inside the faucet housing;
an ultraviolet light source; and
at least one infrared proximity sensor connected to the electrically controlled valve,
wherein an air-gap is provided between a lower end of the aerator and the water outlet opening of the contact protector, and
wherein internal surfaces of the faucet housing and internal surfaces of the contact protector are coated with a photocatalytic material and are exposed to UV-A light from the ultraviolet light source while and after water is flowing through the water pipe.

14. The faucet as in claim 13, wherein the ultraviolet light source comprises a plurality of light emitting diodes with a wavelength between 315 and 400 nm that are mounted circumferentially spaced on an LED carrier fixed between the aerator and the discharge end of the water pipe.

15. The faucet as in claim 14, wherein the ultraviolet light source is coated with a photocatalytic material.

16. The faucet as in claim 13, wherein the contact protector comprises a substantially cylindrical outer wall and a funnel-shaped inner wall section extending downwardly to the water outlet opening, the water opening being arranged inside and above a lower end of the substantially cylindrical outer wall.

17. The faucet as in claim 16, wherein the contact protector is configured with at least one row of circumferentially spaced overflow openings.

18. The faucet as in claim 13, wherein the photocatalytic material is titanium dioxide (TiO₂).

19. A method for providing germ-free water for human consumption, comprising:
providing the water faucet as in claim 10;
detecting the presence of an object, such as hands, under the water outlet end of the housing by the proximity sensor;
activating the ultraviolet light source;
monitoring the operational status of the ultraviolet light source; and
activating the flow of water through the electrically activated valve only if the ultraviolet light source is operational.

20. A method for providing germ-free water for human consumption, comprising:
providing the water faucet as in claim 13;
detecting the presence of an object, such as hands, under the water outlet end of the housing by the infrared proximity sensor;
activating the ultraviolet light source;
monitoring the operational status of the ultraviolet light source; and
activating the flow of water through the electrically controlled valve only if the ultraviolet light source is operational.
